SEQUENCE LISTING (40 sequences)

SEQ ID NO: 1 - DNA, 23 bp, Artificial Sequence
  primer L3b
ggctgagctcggtggtcctggct

SEQ ID NO: 2 - DNA, 39 bp, Artificial Sequence
  primer SMI7
ccagccggccatggctgatgtgcagctggtggagtctgg

SEQ ID NO: 3 - DNA, 39 bp, Artificial Sequence
  primer SMI8
ccagccggccatggctcaggtgcagctggtggagtctgg

SEQ ID NO: 4 - DNA, 33 bp, Artificial Sequence
  primer A4short
catgccatgactcgcggcccagccggccatggc

SEQ ID NO: 5 - DNA, 23 bp, Artificial Sequence
  primer ABL013
gaggtbcarctgcaggastcygg

SEQ ID NO: 6 - DNA, 53 bp, Artificial Sequence
  short hinge primer
aacagttaagcttccgcttgcggccgcggagctggggtcttcgctgtggtgcg

SEQ ID NO: 7 - DNA, 53 bp, Artificial Sequence
  long hinge primer
aacagttaagcttccgcttgcggccgctggttgtggttttggtgtcttgggtt

SEQ ID NO: 8 - DNA, 57 bp, Artificial Sequence
  primer ABL037
catgccatgactcgcggcccagccggccatggccgaggtgcagctggtggagtctgg

SEQ ID NO: 9 - DNA, 57 bp, Artificial Sequence
  primer ABL038
catgccatgactcgcggcccagccggccatggccgatgtgcagctggtggagtctgg

SEQ ID NO: 10 - DNA, 57 bp, Artificial Sequence
  primer ABL039
catgccatgactcgcggcccagccggccatggccgcggtgcagctggtggagtctgg

SEQ ID NO: 11 - DNA, 57 bp, Artificial Sequence
  primer ABL040
catgccatgactcgcggcccagccggccatggccgccgtgcagctggtggattctgg

SEQ ID NO: 12 - DNA, 57 bp, Artificial Sequence
  primer ABL041
catgccatgactcgcggcccagccggccatggcccaggtgcagctggtggagtctgg

SEQ ID NO: 13 - DNA, 57 bp, Artificial Sequence
  primer ABL042
catgccatgactcgcggcccagccggccatggcccaggtacagctggtggagtctgg

SEQ ID NO: 14 - DNA, 57 bp, Artificial Sequence
  primer ABL043
catgccatgactcgcggcccagccggccatggcccaggtaaagctggaggagtctgg

SEQ ID NO: 15 - DNA, 23 bp, Artificial Sequence
  set 1 primer 1 in table 2
cagrtgcagctggtgcartctgg

SEQ ID NO: 16 - DNA, 23 bp, Artificial Sequence
  set 1 primer 2 in table 2
saggtccagctggtrcagtctgg

SEQ ID NO: 17 - DNA, 23 bp, Artificial Sequence
  set 2 primer 1 in table 2
saggtgcagctggtggagtctgg

SEQ ID NO: 18 - DNA, 23 bp, Artificial Sequence
  set 2 primer 2 in table 2
gargtgcagctggtgcagtctgg

SEQ ID NO: 19 - DNA, 23 bp, Artificial Sequence
  set 3 primer 1 in table 2
cagstgcagctgcaggagtcsgg

SEQ ID NO: 20 - DNA, 23 bp, Artificial Sequence
  set 3 primer 2 in table 2
caggtacagctgcagcagtcagg

SEQ ID NO: 21 - DNA, 23 bp, Artificial Sequence
  primer 4 in table 2
cagrtcaccttgaaggagtctgg

SEQ ID NO: 22 - DNA, 23 bp, Artificial Sequence
  primer 5 in table 2
caggtgcagctgcagcagtgggg

SEQ ID NO: 23 - DNA, 24 bp, Artificial Sequence
  IgG-specific primer
gtccaccttggtgttgctgggctt

SEQ ID NO: 24 - DNA, 24 bp, Artificial Sequence
  IgM-specific primer
tggaagaggcacgttcttttcttt

SEQ ID NO: 25 - DNA, 23 bp, Artificial Sequence
  Table 3 Vlambda amplification - set 1 primer 1
cagtctgtgytgackcagccrcc

SEQ ID NO: 26 - DNA, 23 bp, Artificial Sequence
  Table 3 Vlambda amplification - set 1 primer 2
cwgcctgtgctgactcagccmcc

SEQ ID NO: 27 - DNA, 21 bp, Artificial Sequence
  Table 3 Vlambda amplification - set 1 primer 3
cagtctgccctgactcagcct

SEQ ID NO: 28 - DNA, 23 bp, Artificial Sequence
  Table 3 Vlambda amplification - primer 2
cagcytgtgctgactcaatcryc

SEQ ID NO: 29 - DNA, 23 bp, Artificial Sequence
  Table 3 Vlambda amplification - set 3 primer 1
caggctgtgctgactcagccgkc

SEQ ID NO: 30 - DNA, 23 bp, Artificial Sequence
  Table 3 Vlambda amplification - set 3 primer 2
caggcagggctgactcagccacc

SEQ ID NO: 31 - DNA, 23 bp, Artificial Sequence
  Table 3 Vlambda amplification - primer 4
tcctatgagctgacwcagccacc

SEQ ID NO: 32 - DNA, 23 bp, Artificial Sequence
  Table 3 Vlambda amplification - primer 5
aattttatgctgactcagcccca

SEQ ID NO: 33 - DNA, 23 bp, Artificial Sequence
  Table 3 Vlambda amplification - set 6 primer 1
cagrctgtggtgacycaggagcc

SEQ ID NO: 34 - DNA, 23 bp, Artificial Sequence
  Table 3 Vlambda amplification - set 6 primer 2
tcttctgagctgactcaggaccc

SEQ ID NO: 35 - DNA, 23 bp, Artificial Sequence
  Table 3 Vkappa amplification - set I primer 1
gaaattgtgwtgacrcagtctcc

SEQ ID NO: 36 - DNA, 23 bp, Artificial Sequence
  Table 3 Vkappa amplification - set I primer 2
gaaattgtgctgactcagtctcc

SEQ ID NO: 37 - DNA, 23 bp, Artificial Sequence
  Table 3 Vkappa amplification - set II primer 1
gatgttgtgatgactcagtctcc

SEQ ID NO: 38 - DNA, 23 bp, Artificial Sequence
  Table 3 Vkappa amplification - set II primer 2
gayatygtgatgacccagwctcc

SEQ ID NO: 39 - DNA, 23 bp, Artificial Sequence
  Table 3 Vkappa amplification - primer III
gacatccagwtgacccagtctcc

SEQ ID NO: 40 - DNA, 23 bp, Artificial Sequence
  Table 3 Vkappa amplification - primer IV
gaaacgacactcacgcagtctcc

(19) United States
(12) Patent Application Publication
(10) Pub. No.: US 2005/0037358 A1
(43) Pub. Date: Feb. 17, 2005

(54) Method for cloning of variable domain sequences

(76) Inventor: Serge Muyldermans, Hoeilaart (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/499,500

(22) PCT Filed: Dec. 20, 2002
(86) PCT No.: PCT/EP02/14662

(30) Foreign Application Priority Data
Dec. 21, 2001 (EP) 01205100.9

(51) Int. Cl. C12Q 1/68; C07H 21/04; C12P 19/34; A61K 39/395; C07K 16/30; C12N 5/06

(52) U.S. Cl. 435/6; 435/69.1; 435/91.2; 435/320.1; 435/326; 530/388.1; 536/23.53

(57) ABSTRACT

A method for cloning immunoglobulin variable domain sequences derived from immunoglobulins, and repertory library of immunoglobulin variable domain sequences made according to the method are disclosed.

### FIELD OF THE INVENTION

The present invention relates to the cloning of variable domain polynucleotide sequences derived from immunoglobulins.

### BACKGROUND OF THE INVENTION

Immunoglobulin (Ig) chains are divided into a number of domains. At the N-terminal end of an Ig chain is a variable domain. The variable domains on the heavy and light chains fit together to form a binding site designed to receive a particular antigen. The variable domains are so called because their amino acid sequences vary particularly from one molecule to another. This variation in sequence enables the molecules to recognise an extremely wide variety of targets. Each variable domain comprises a number of areas of relatively conserved sequence and also three areas of hypervariable sequence. The three hypervariable areas are known as complementarity determining regions (CDRs). It has been discovered that isolated immunoglobulin variable domains (IGVDs), such as for example, heavy chain variable domains (HCVDs) can bind to antigen in a 1:1 ratio and with binding constants of equivalent magnitude to those of complete antibody molecules. Since these IGVDs can have binding affinities similar to that of complete Ig molecules, they can be used in many of the ways as are Ig molecules or fragments thereof. For example, Ig molecules are currently used in therapy, in diagnosis, in vaccination, in modulation of activities of hormones or growth factors, in detection, in biosensors and even in catalysis. It is envisaged that the small size of the IGVDs may confer some advantages over complete antibodies, for example, in neutralising the activity of low molecular weight drugs and allowing their filtration from the kidneys with drug attached, in penetrating tissues and tumours, in neutralising viruses by binding to small conserved regions on the surfaces of viruses, in high resolution epitope mapping of proteins and in vaccination by IGVDs which mimic antigens. It is said that a mixture of all or most of the IGVDs derived from an individual forms a repertoire. Repertoire cloning of variable domains is described in the art. The latter method is fully described in European Patent number 0 368 684. Essentially, said method for repertoire cloning employs the polymerase chain reaction and needs two species-specific primers, annealing on conserved DNA sequences flanking the variable domains, for cloning. Cloning into a suitable vector is facilitated by the incorporation of a restriction enzyme site into the two species specific primers.

Patent application number WO 99/23221, granted patent numbers EP 0 368 684 and U.S. Pat. No. 6,291,161, Van der Linden et al (J. Immunol Methods, 240, p185 to 195) and Larrick J W et al (Progress in Biotechnology, 5, p231 to 246) disclose methods for isolating genes encoding IGVDs and cite the use of two species specific primers which flank the IGVD region. Using two species-specific primers requires fore-knowledge of the sequences of the regions flanking both ends of the IGVD for every species. For some species, for example, llama, IgG sequence information is not readily available. Where sequence information is not available, primers that are not precisely complementary for that species are commonly used, so leading to less efficient primer annealing and a consequently smaller repertory diversity. In fact, the use of primers not precisely complementary to the target results in forced mutations in the repertory library so produced. It has been shown that forced mutations influence the functionality of the IGVDs, therefore, a method that reduces the number of primer-forced mutations would significantly increase the size of a functional repertory library. See for example Kipriyanov S M et al, Two amino acid mutations in an anti-human CD3 single chain Fv antibody fragment that affect the yield on bacterial secretion but not the affinity, Protein Eng. (1997),10(4), p445-53; de Haard H, et al, Vernier zone residue 4 of mouse subgroup II kappa light chains is a critical determinant for antigen recognition, Immunotechnology, (1999), 4(3-4), p203-15; de Haard H J, et al, Absolute conservation of residue 6 of immunoglobulin heavy chain variable regions of class IIA is required for correct folding, Protein Eng (1998),11(12), p1267-76; Honegger A, Pluckthun A. J, The influence of the buried glutamine or glutamate residue in position 6 on the structure of immunoglobulin variable domains, Mol Biol (2001), 309(3), p687-99; Jung S, et al, The importance of framework residues H6, H7 and H10 in antibody heavy chains: experimental evidence for a new structural subclassification of antibody V(H) domains, J Mol Biol (2001), 309(3), p701-16; Langedijk A C, et al, The nature of antibody heavy chain residue H6 strongly influences the stability of a VH domain lacking the disulfide bridge, J Mol Biol (1998), 283(1), p95-110.

Patent application number WO 01/79481 discloses a method for constructing a library of VH genes using a method to amplify the product of a poly-dT-primed cDNA synthesis. At the 3′ end of the gene, a species-specific primer that anneals to the constant region is used. At the 5′ end of the gene, a synthetic tail is added to the start of the gene by using RT Cap Extension. Subsequent steps are required to remove the non-coding regions and to create restriction enzyme sites required for cloning. Apart from involving a large number of procedural steps, the method uses several consecutive DNA polymerizations which are known to introduce unwanted mutations and to decrease the yield of library diversity.

The method of the present invention is an alternative method for repertory cloning of IGVDs and starts from a sample comprising messenger RNA. This novel method uses only one species-specific primer which anneals to a sequence located at or adjacent to the 3′ end of the antisense strand of the IGVD sequence after first strand cDNA synthesis from mRNA. The double stranded DNA so produced encompasses the IGVD sequence and all of the constant region. By taking advantage of a naturally occurring restriction site positioned such that cleavage with a restriction enzyme directed thereto produces double stranded DNA encoding at least part of an IGVD sequence, the IGVD fragment so produced can be conveniently cloned and expressed. The use of a single species-specific primer in combination with the naturally occurring restriction site achieves a higher library diversity because it is less dependent on sequence variation from species to species.

Furthermore, the use of the naturally-occurring restriction site results in no primer-forced mutations at the 3′-end.

The method also represents a significant cost-time saving over methods of the art because the need to optimise the annealing of the 3′-end primer for every species is obviated, and the number of procedural steps is appreciably reduced.

### AIMS AND DETAILED DESCRIPTION OF THE INVENTION

The present invention relates to an efficient method for the cloning of immunoglobulin variable domain (IGVD) sequences. At the level of the gene, it is well known that heavy chains are encoded by a “rearranged” gene which is built from three gene segments: an “unrearranged” VH gene (encoding the N-terminal three framework regions, first two complete CDRs and the first part of the third CDR), a diversity (DH)segment (encoding the central portion of the third GDR) and a joining segment (JH) (encoding the last part of the third CDR and the fourth framework region). In the maturation of B-cells, the genes rearrange so that each unrearranged VH gene is linked to one DFI gene and one JH gene. The rearranged gene corresponds to VH-DHJH. This rearranged gene is linked to a gene which encodes the constant portion of the Ig chain. A repertoire of IGVD consisting of at least part of the variable heavy domain of a molecule from the immunoglobin superfamily is an end product of processes involving methods according to the present invention. Alternatively, a repertoire of IGVD consisting of at least part of the light chain variable domain of a molecule from the immunoglobin superfamily is an end product of processes involving methods according to the present invention. Alternatively, a repertoire of IGVD consists of at least part of the heavy chain variable domain of a molecule from the immunoglobin superfamily and at least part of the variable light domain of a molecule from the immunoglobin superfamily. The term “repertoire” in relation to immunoglobins means a range of differing antibody specificities which approximates to or resembles that seen in an animal.

In a first embodiment the invention provides a method for cloning IGVD polynucleotide sequences, said method comprises: (a) providing a sample comprising mRNA, (b) carrying out a first strand cDNA synthesis, (c) producing double stranded DNA by use of a first primer that is capable of hybridizing to a site at or adjacent to the start of the IGVD on the antisense strand, (d) cleaving said double stranded DNA with a restriction enzyme specific for a restriction site positioned such that cleavage with the restriction enzyme directed thereto produces double stranded DNA encoding a functional IGVD fragment (e) cloning the resulting IGVD sequences into a vector.

According to the invention, a IGVD polynucleotide sequence is a heavy chain variable domain (HCVD) polynucleotide sequence or a light chain variable domain (LCVD) polynucleotide sequence. A repertoire of IGVD polynucleotide sequences comprises HCVD polynucleotide sequences and/or light chain variable domain polynucleotide sequences.

The fragment of IGVD double stranded DNA generated according to the cleaving of step (d) may contain less, more or exactly the number of nucleotide residues of full length IGVD, however, in all cases the fragment generated is capable of binding to antigen.

Thus a method of the present invention can start from isolated mRNA. mRNA may be isolated in a known manner from a cell or cell line which is preferentially known to produce immunoglobulins. mRNA may be separated from other RNA by oligo-dT chromatography or other methods known in the art. A complementary strand of cDNA may then be synthesized on the mRNA template, using reverse transcriptase and a suitable primer (called herein a “universal primer”), to yield a cDNA/mRNA heteroduplex. A suitable universal primer comprises an oligo-dT or alternatively can comprise a set of random primers.

Double stranded DNA is made from the cDNA/mRNA heteroduplex by using a species-specific primer. According to a method of the invention, the species specific primer can be a single species-specific primer, or a mixture of species-specific primers. The species-specific primer anneals to a sequence located at or adjacent to the 3′ end of the antisense strand of the IGVD sequence. The term “at or adjacent” means that the primer anneals to a polynucleotide sequence that encodes the N-terminal end of the IGVD sequence. Ideally the primer anneals “at” the 3′ end of the anti-sense strand of the IGVD sequence. Optionally the primer anneals “adjacent” to the 3′ end of the anti-sense strand of the HCVD sequence, meaning that extra DNA, not belonging to the IGVD sequence is also cloned at the 5′-end of the sense strand. Annealing of said primer(s) occurs under conditions which allow said primer(s) to hybridise to the nucleic acid. The term “species-specific” means here that the primers are designed to anneal with sequences at or adjacent to the 3′ end of the anti-sense strand of the IGVD sequences of one particular species, e.g. mouse, human, camelid-species. Furthermore, said species-specific primer may be one single primer having a consensus polynucleotide sequence derived from all the families of heavy chain variable region genes but may also consist of a plurality of primers having a variety of sequences designed to be complementary to the various families of IGVD sequences known. Since the primers may not have a sequence exactly complementary to the target sequence to which it is to be annealed, for instance because of nucleotide variations or because of the introduction of a restriction enzyme recognition site, it may be necessary to adjust the conditions in the annealing mixture to enable the primers to anneal to the double stranded nucleic acid. This procedure is well known to the person skilled in the art. Advantageously, the species-specific primer comprises a sequence including a restriction enzyme recognition site. The sequence recognized by the restriction enzyme does not need to be in the part of the primer which anneals to the double stranded nucleic acid, but may be provided as an extension which does not anneal. The use of a primer or a combination of primers with one or more restriction sites has the advantage that the DNA can be cut with at least one restriction enzyme which can leave 3′ or 5′ overhanging nucleotides or blunted ends. An important element of the present invention is that the isolation of IGVD sequences occurs with only one species specific primer.

The double stranded cDNA produced using a species-specific primer according to the invention comprises the region between the species-specific primer and the site used to prime cDNA synthesis from mRNA. It thus comprises at least the IGVD and all of the constant region. The double stranded cDNA so produced may be used for cloning as described below. Alternatively, it may be amplified prior to cloning using the species specific primer, and a second primer that binds to a site downstream from the 3′ end of the sense strand of the IGVD sequence and that is not species-specific. The second primer can comprise a sequence that anneals to a consensus region downstream of the 3′ end of the sense strand of the IGVD sequence, and that is present across all species (i.e. is not species specific). Alternatively the second primer comprises the sequence that is used to prime the synthesis of cDNA from mRNA (the universal primer) according to the invention. Where cDNA synthesis is primed using a set of random primers, the second primer comprises a mixture of said random primers. Alternatively, the second primer is a sequence that comprises oligo-dT.

The double stranded cDNA may be amplified according to methods known in the art. In one example, the amplification method comprises the following steps: (a) denaturing the sample comprising cDNA to separate the two strands, (b) annealing to said sample the species-specific primer and a second primer, under conditions which allow said primers to hybridise to the nucleic acid, (c) adding to the annealed sample a DNA polymerase enzyme in the presence of deoxynucleoside triphosphates under conditions which cause primer extension to take place and (d) denaturing the sample under conditions such that the extended primers become separated from the sequence. Preferably, the method further includes step (e) wherein steps (b) to (d) are repeated a plurality of times.

The denaturing step (d) may for example be carried out by heating the sample, by use of chaotropic agents, such as urea or guanidine, or by the use of changes in ionic strength of pH. Preferably, denaturing is carried out by heating since this is readily reversible. Where heating is used to carry out the denaturing, it will be usual to use a thermostable DNA polymerase since this will not need to be replenished at each cycle. The product, double stranded cDNA, may be separated from the mixture by for instance gel electrophoresis using agarose gels. Alternatively the double stranded cDNA may be used without purification and cloned according to the methods described below.

After amplification, the double stranded cDNA produced using a specific-specific primer and a second primer according to the invention comprises at least IGVD and part of the constant region.

In an alternative embodiment of the invention, the double stranded cDNA is made from the cDNA/mRNA heteroduplex by a DNA amplification step. The template for the amplification is the cDNA/mRNA duplex formed after first strand cDNA synthesis from a suitable universal primer. The primers used to amplify the template are the species-specific primer and the second primer as described above. The cDNA/mRNA heteroduplex may be amplified according to methods known in the art or according to the example described above. After amplification, the double stranded cDNA produced using a specific-specific primer and a second primer according to the invention comprises at least IGVD and part of the constant region.

The inventors have surprisingly found that when the unique restriction site present (in a rearranged IGVD) at the junction of the IGVD and constant region (more precisely at the 3′-end of the framework IV (4) region) is utilized for cloning, the diversity of the library so produced is a significant advancement on the prior art. In humans and camelids a suitable restriction site has been found to be the BstEII-recognition site. Thus, the double stranded cDNA produced according to the invention may be cleaved using appropriate restriction enzymes, for example, BstEII in the case of humans and camelids, and the restriction enzyme that is encoded by the species-specific primer. As an optional step, the resulting restriction fragments can be separated and isolated by agarose gel electrophoresis, for example. Preferably the choice of the restriction site is such that the double stranded cDNA can be conveniently cloned into an expression vector, such that the functional IGVD can be expressed.

It is part of the invention to take advantage of other restriction sites positioned in the double stranded DNA such that cleavage with the restriction enzyme directed thereto produces double stranded DNA encoding a functional IGVD fragment. An example of a suitable restriction site is BstEII, as described above. Other restriction sites may be used according to the invention. Sites may be screened by persons skilled in the art using known techniques. For example, a repertory library of double stranded DNA generated according to the invention may be screened for suitable restriction sites using a binding assay and a collection of restriction enzymes. The fragments generated after digestion are cloned and tested for binding. The presence of one or more suitably located restriction sites are indicated by a cleavage product which expresses a functional IGVD fragment. The restriction site is located towards the 3′ end of the IGVD, preferably at the junction between the IGVD and constant region. The fragment of IGVD double stranded DNA generated after cleavage by said restriction site may contain less, more or exactly the number of nucleotide residues of full length IGVD, however, in all cases the fragment generated is capable of binding to antigen.

Alternatively the method of the present invention for repertoire cloning of IGVDs can be carried out starting from a sample comprising cDNA. Said cDNA is preferentially derived from lymphocytes. Methods for making cDNA from mRNA are well known to the person skilled in the art. The reverse transcription of the first (antisense) strand can be performed in any manner with any suitable universal primer. See, for example, de Haard H J, et al (1999), A large non-immunized human Fab fragment phage library that permits rapid isolation and kinetic analysis of high affinity antibodies J Biol Chem 274, 18218-18230. The cDNA may be amplified using species-specific primers and a second primer, such as a universal primer as described previously. The product may be separated from the mixture by gel electrophoresis, for example. Alternatively, it may be used without purification and inserted directly into a suitable cloning vector. Either way, use is made of the unique restriction between the IGVD and the constant region as described previously.

In an alternative method the use of a species specific primer can be omitted. After cDNA synthesis using a universal primer, synthetic sequences (also called adaptor sequences) may be attached to the 5′ end of the DNA strand by various methods well known for ligating DNA sequences together. RT CapExtension is only one example and is described in patent application number WO 01/179481 which is herein incorporated by reference. Conveniently the synthetic sequences or adaptor sequences comprise one or more restriction sites that can be used for cloning. In this way a repertoire of, for example, human or camelid variable heavy chains can be isolated by cleavage with BstEII (which resides in framework 4) and a restriction enzyme which is encoded by the adaptor ligated to the cDNA. The resulting restriction fragments can be separated and isolated by agarose gel electrophoresis, for example, and subsequently cloned in a suitable vector. Thus, an alternative method provides a technique for cloning human or camelid immunoglobulin IGVD sequences comprising (1) providing a sample comprising mRNA, (2) carrying out a cDNA synthesis, (3) ligating an adaptor sequence comprising at least one restriction enzyme to the 5′ end of the DNA, (3a) optionally amplifying the sequence using the adapter sequence and the universal sequence as primers, (4) cleaving the resulting DNA with BstEII and a restriction enzyme encoded by said adaptor and (5) cloning the resulting human or camelid IGVD sequences into a vector.

A “vector” as mentioned herein is any genetic element, e.g. a plasmid chromosome, a virus, behaving either as an autonomous unit of polynucleotide replication within a cell (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, bacteriophages and cosmids. “Expression vectors” may contain polynucleotide sequences which are necessary to effect ligation or insertion of the vector into desired host cell and to effect the expression of the attached segment. Such sequences differ depending on the host organism; they include promoter sequences, to effect transcription, enhancer sequences to increase transcription, ribosomal binding site sequences and transcription and translation termination sequences. Alternatively, expression vectors may be capable of directly expressing gene products, such as a repertoire of variable heavy chain products encoded therein without ligation or integration of the vector into host cell DNA sequences.

In a particular embodiment the sample comprising mRNA is derived from lymphocytes which have been stimulated to enhance the production of mRNA. Lymphocytes, and particularly B-lymphocytes, are able to synthesize immunoglobulins and these cells generally possess mRNA that can be translated in immunoglobulins chains. Lymphocytes can be derived from immunized or non-immunized animals. In general sources of mRNA can comprise peripheral blood cells, bone marrow cells, spleen cells or lymph node cells (such as B-lymphocytes or plasma cells), patients suffering from at least one autoimmune disorder or cancer, patients suffering from autoimmune diseases such as systemic lupus erythematosus, systemic sclerosus, rheumatoid arthritis, antiphospholipid syndrome or vasculitis.

In another embodiment the first strand cDNA synthesis that forms part of the method of the present invention can be carried out via random priming or via oligo-dT-priming. Both priming methods are well known in the art and do not need further explanation.

In another embodiment the species-specific primer encodes for at least one restriction enzyme. Thus at least one restriction enzyme site can be encoded by a sequence comprising the primer, wherein said restriction enzyme site(s) does not need to anneal with the 3′ end on the anti-sense strands of each of the IGVD sequences.

In another embodiment the IGVD can be derived from animals of the camelid family. In said family immunoglobulins devoid of light polypeptide chains are found. IGVD sequences derived from camelids are therefore HCVDs and are designated as VHH's. “Camelids” comprise old world camelids (Camelus bactrianus and Camelus dromaderius) and new world camelids (for example Lama paccos, Lama glama, Llama guanacoe and Lama vicugna). European Patent number 0 656 946 describes the isolation and uses of camelid immunoglobulins and is incorporated herein by reference.

Another embodiment the method of the present invention provides an expression library comprising a repertoire of IGVD polynucleotide sequences. In another embodiment the method of the present invention, said IGVD polynucleotide sequences are HCVD polynucleotide sequences. In another embodiment the method of the present invention, said IGVD polynucleotide sequences are LCVD polynucleotide sequences. In another embodiment the method of the present invention, said IGVD polynucleotide sequences are HCVD polynucleotide sequences and LCVD polynucleotide sequences. Thus, the products obtained by the present invention, cDNA encoding IGVD sequences, can be cloned directly into an expression vector. The host may be prokaryotic or eukaryotic, but is preferably bacterial. Preferably, the choice of the restriction enzyme site in the species-specific primer and in the vector, and other features of the vector will allow the expression of complete IGVD sequences.

If desired, a gene for an IGVD can be mutated to improve the properties of the expressed IGVD, for example to increase the yields of expression or the solubility of the IGVD, to improve the affinity of the IGVD or to introduce a second site for covalent attachment or non-covalent attachment of other molecules. In particular it would be desirable to introduce a second site for binding to molecules with effector functions, such as components of complement, or receptors on the surfaces of cells. Thus, hydrophobic residues which would normally be at the interface of the IGVD with the light chain variable domain could be mutated to more hydrophilic residues to improve solubility; residues in the CDR loops could be mutated to improve antigen binding; residues on the other loops or parts of the beta-sheet could be mutated to introduce new binding activities. Mutations could include single point mutations, multiple point mutations or more extensive changes and could be introduced by any of a variety of recombinant DNA methods, for example gene synthesis, site directed mutagenesis or the polymerase chain reaction. Thus, in another embodiment the method of the present invention may be used to make variations in the sequences encoding the IGVDs. For example, this may be achieved by using mutagenic nucleotide triphosphates during the amplification step such that point mutations are scattered throughout the target region. Alternatively, such point mutations are introduced by performing a large number of cycles of amplification, as errors due to the natural error rate of the DNA polymerase are amplified, particularly when using high concentrations of nucleoside triphosphates.

The basic techniques for manipulating Ig molecules by recombinant DNA technology are extensively described in the art (see for example: Antibody Engineering, A practical approach, ed. J. McCafferty, H. R. Hoogenboom and D. J. Chiswell).

One embodiment of the present invention is a method for cloning polynucleotide sequences encoding immunoglobulin variable domains (IGVD):

(a) providing a sample comprising mRNA,

(b) carrying out a first strand cDNA synthesis using a universal primer,

(c) carrying out a second strand DNA synthesis using a first primer capable of hybridising to a site at or adjacent to the 3′ end of each of the IGVD sequences on the anti-sense strand so producing double stranded DNA,

(d) cleaving the double stranded DNA with a restriction enzyme specific for a restriction site positioned such that cleavage with the restriction enzyme directed thereto produces double stranded DNA encoding a functional IGVD fragment, and

(e) cloning the resulting variable domain fragment sequences into a vector.

Another embodiment of the present invention is a method as defined above wherein the double stranded DNA produced in step (c) is subsequently amplified using said first primer and said universal primer.

Another embodiment of the present invention is a method as defined above wherein step (c) is an amplification step comprising use of said first primer and said universal primer, and the product of step (b) as the template.

Another embodiment of the present invention is a method as defined above wherein the universal primer comprises the sequence of oligo-dT.

Another embodiment of the present invention is a method as defined above wherein the universal primer comprises the sequence of a set of random primers.

Another embodiment of the present invention is a method as defined above, wherein said first primer encodes for at least one enzyme restriction site.

Another embodiment of the present invention is a method as defined above wherein said sample comprises mRNA derived from lymphocytes.

Another embodiment of the present invention is a method as defined above wherein the restriction site of step (d) is BstEII.

Another embodiment of the present invention is a method as defined above, wherein said mRNA is derived from humans.

Another embodiment of the present invention is a method as defined above, wherein said mRNA is derived from camelids.

Another embodiment of the present invention is a method as defined above wherein said vector is an expression vector able to express at least part of IGVD polynucleotide sequences.

Another embodiment of the present invention is a method as defined above wherein said IGVD polynucleotide sequences are heavy chain variable domain polynucleotide sequences.

Another embodiment of the present invention is a method as defined above wherein said IGVD polynucleotide sequences are light chain variable domain polynucleotide sequences.

Another embodiment of the present invention is a method as defined above wherein said IGVD polynucleotide sequences are heavy chain variable domain and light chain variable domain polynucleotide sequences.

Another embodiment of the present invention is an expression library obtainable by a method as defined above comprising a repertoire of IGVD polynucleotide sequences.

Another embodiment of the present invention is an expression library obtained by a method as defined above comprising a repertoire of IGVD polynucleotide sequences.

Another embodiment of the present invention is an IGVD polynucleotide obtainable according to the methods as defined above.

Another embodiment of the present invention is an IGVD polynucleotide obtained according to the methods as defined above.

Another embodiment of the present invention is a diagnostic assay based on the use of an expression library as defined above, or an IGVD polynucleotide as defined above.

Another embodiment of the present invention is a diagnostic report obtained from the diagnostic assay as defined above.

Another embodiment of the present invention is a use of a polypeptide obtained after expression of one of the cloned sequences as defined above for the manufacture of a medicament.

### FIGURES

FIG. 1. Comparison of the titres of phage prepared using a single species-specific primer, and using two species specific primer, according to Example 2. Key: -▴- two IgG derived primers; ---▴--- experimental blank, two primer method; -▪- a single IgG primer combined with oligo-dT; ---▪--- experimental blank, one primer method.

FIG. 2. Agarose electophoresis gel showing the amplification of two fragments (1650 and 1300) resulting from a VHH cDNA repertoire according to Example 2.

FIG. 3. Agarose electophoresis gel showing the results of a restriction digest with BstEII. Over 90% of amplified fragments contain an internal BstEII site according to Example 2.

### EXAMPLES

### 1. Creating a Repertoire Library of Anti-potyvirus Y Coat Protein VHH

### a. Immunisation

Potyvirus Y coat protein, carboxyterminally linked to a hexahistidine peptide (PVYCP-His₈) was recombinantly expressed in Escherichia coli. At day 1, dromedary ‘48’ was injected with 1 mg of PVYCP-His₆ in Freund's complete adjuvant. At days 8, 15, 22, 29, and 36 a dose of 1 mg PVYCP-His₆ in Freund's incomplete adjuvant was injected. One week after the last PVYCP-His₆ boost, 50 ml of blood was collected from the immunised dromedary.

### b. Isolation of Iymphocytes, mRNA and cDNA Preparation

Peripheral blood lymphocytes (PBL's) were isolated on UNI-SEP MAXI tubes (Wak Chemie Medica) according to the manufacturer's protocol, divided into aliquots of 10⁷ cells, and stored at −80° C. mRNA was isolated from 10⁷ PBL's using the Quickprep Micro mRNA Purification Kit (Amersham Pharmacia Biotech). This mRNA was used as a template in a RT-PCR using primer oligo-dT to synthesise the first strand of cDNA (Ready-To-Go Kit, Amersham Pharmacia Biotech).

### c. Construction of the Library

The Expand High Fidelity PCR System (Roche) was used in all following PCR amplifications and each time a ‘hot start’ was performed by adding the polymerase during the third minute of the first three minutes of denaturing. To amplify the VHH repertoire, three consecutive PCR amplifications were performed. In a first PCR (PCR1) with primers L3b (5′-GGCTGAGCTCGGTGGTCCTGGCT-3′ (SEQ ID NO: 1), annealing to the IgG leader sequence) and oligo-dT (annealing to the polyA sequence which is located downstream of the IgG coding sequences), 2 μl of the synthesised dromedary cDNA was used as template. The template was denatured for 3 minutes at 94° C., followed by 33 cycles of 20 seconds denaturing at 94° C., 1 minute of primer annealing at 52° C. and an elongation step of 3 minutes at 72° C. The amplification was completed with an additional elongation at 72° C. for 10 minutes. VHHs were separated from VHs by 1.2% agarose gel electrophoresis. The fragments corresponding to VHHs (expected size of 1.2-1.3 kb) were excised from gel, purified with the Qiaquick Gel Extraction Kit (Qiagen) and the DNA concentration was determined. A NcoI restriction site (bold in primer sequence) was introduced at the 5′ end in a nested PCR (PCR2), using 5 picogram of purified template from PCR1 with an equimolecular mixture of:

| | |
|---|---|
| SMI7 | |
| (5′-CCAGCCGGCCATGGCTGATGTGCAGCTGGTGG | (SEQ ID NO: 2) |
| AGTCTGG-3′) and | |
| SMI8 | |
| (5′-CCAGCCGGCCATGGCTCAGGTGCAGCTGGTGG | (SEQ ID NO: 3) |
| AGTCTGG-3′) | |

as the upstream primers and ologo-dT as the downstream primer. The template was denatured for 3 minutes at 94° C., followed by 25 cycles of 20 seconds at 94° C., 1 minute at 48° C. and 3 minutes at 72° C. The amplification was completed with an additional elongation step at 72° C. for 10 minutes. The amplified 1.2-1.3 kb 20 fragments were gel-purified (Qiaquick Gel Extraction Kit) and the DNA concentration was determined.

To introduce a SfiI restriction site (bold in primer sequence) at the 5′ end, a third PCR (PCR3) was performed, with A4short (5′-CATGCCATGACTCGCGGCCCAGCCGGCCATGGC-3′) (SEQ ID NO: 4) as the upstream primer and oligo-dT using 5 μg of the PCR2 purified as the template. The experimental conditions for this PCR were identical as for PCR2. The amplified fragments resulting from PCR3 were purified with the Qiaquick PCR purification Kit (Qiagen). Approximately 5 pg of PCR3 amplification product was doubly digested with SfiI and BstEII, the latter restriction site being present in framework 4 of the dromedary VHHS. Restriction fragments were separated by agarose gel electrophoresis and fragments with an approximate size of 380 bp were excised and purified with the Qiaquick Gel Extraction Kit. Approximately 350 ng of SfiI-BstEII digested VHH repertoire was ligated into 1200 ng of the corresponding restriction sites of phagemid pHEN4 (Ghahroudi et at. 1997), using 2 μl of the highly concentrated T4 DNA ligase (20 units/μl, Promega) in a total volume of 500 μl. After an overnight incubation at 140° C., the ligation reaction was purified by a double phenol and a subsequent chloroform extraction. DNA was precipitated by adding 0.1 volume of 5M LiCl and 2.5 volume of cold 100% ethanol followed by a 30-minute −20° C. incubation. DNA was pelleted and washed with 70% ethanol. The DNA pellet was air-dried and dissolved in 80 μl of water. Twelve transformations were performed in 0.1 cm cuvettes using the E. coli pulser (Biorad) at 2.5 MΩ and 1.8 kV with 5 μl (each containing an equivalent of 50 ng of vector) of purified ligated construct mixed with freshly prepared TG1 electrocompetent cells (Sambrook and Russell 2001 Molecular Cloning A laboratory manual third edition, Cold spring harbor Laboratory press Cold spring harbor, New York, Page 1.120-1.121). We used 1 ml of SOC medium for each electroporation to recover the transformed TG1 cells. The transformed TG1 cells were incubated for 1 h at 37° C. under moderate shaking. Selection of pHEN4-harboring TG1 cells was done on LB-Ap¹⁰⁰-2% glucose plates. A library of 10⁹ individual transformants was obtained. By colony PCR using a matching framework1 and framework4 primer, we verified the presence of insert-containing clones of the library. Out of 93 tested individual colonies, all contained an insert with a fragment size corresponding a framework1-framework4 amplified VHH.

### d. Isolation of a PVYCP Specific.Binder by Phase Display and Biopanning

Cloning of the VHH repertoire in pHEN4 allowed us to express a library of single VHHs as fusion proteins with pill on the tip of phage M13. Rescue of the library and selection of binders in immunotubes coated with PVYCP-His₆ (100 μg/ml) was performed as described by Ghahroudi et al. (1997), FEBS Letters 414:521-526. After the second round of panning, we were able to isolate PVYCP-specific binders.

### 2. Comparing the Library Diversity Obtained using a Single Species-specific Primer, with that Obtained using Two Species-specific Primers

### a. Immunization

A llama (Llama glama) was immunized with the human targets IgE, carcinoembryonic antigen (CEA), von Willebrand factor (vWF) and interleukin-6 (IL-6). For immunization, the targets were formulated as an emulsion with an appropriate, animal-friendly adjuvant (Specoll, CEDI Diagnostics B.V.). The antigen cocktail was administered by double-spot injections intramuscularly in the neck. The animal received 6 injections of the emulsion, containing between 100 and 25 μg of each antigen at weekly intervals. At different time points during immunization, 10 ml blood samples were collected from the animal and sera were prepared. The induction of an antigen specific humoral immune response was verified using the serum samples in an ELISA experiment with the targets as immobilized antigen. Five days after the last immunization, a blood sample of 150 ml was collected. From this sample, conventional and heavy-chain antibodies (HcAbs) were fractionated (Lauwereys M, et al (1998) Potent enzyme inhibitors derived from dromedary heavy-chain antibodies. EMBO J 17,3512-3520.) and used in an ELISA, which revealed that the HcAbs were responsible for the antigen specific humoral immune response. Peripheral blood lymphocytes (PBLs), as the genetic source of the llama heavy chain immunoglobulins, were isolated from the 150 ml blood sample using a Ficoll-Paque gradient (Amersham Biosciences) yielding 5×10⁸ PBLs. The maximal diversity of antibodies is expected to be equal to the number of sampled B-lymphocytes, which is about 10% of the number of PBLs (5×10⁷). The fraction of heavy-chain antibodies in llama is up to 20% of the number of B-lymphocytes. Therefore, the maximal diversity of HcAbs in the 150 ml blood sample is calculated as 10⁷ different molecules. Total RNA (around 400 μg) was isolated from these cells using an acid guanidinium thiocyanate extraction (Chomczynski P and Sacchi N (1987). Single-step method of RNA isolation by acid guanidinium thiocyanate-phenol-chloroform extraction. Anal Biochem 162, 156-159.).

### b. Construction of Immune Libraries

### i) Amplification of Repertoire with Two IgG-derived Primers

cDNA was prepared on 100 μg total RNA with M-MLV Reverse Transcriptase (Gibco BRL) and a hexanucleotide random primer (Amersham Biosciences) as described before (de Haard et al., 1999). The cDNA was purified with a phenol/chloroform extraction combined with an ethanol precipitation and subsequently used as template to specifically amplify the VHH repertoire. The repertoire was amplified in a hinge-dependent approach using two IgG specific oligonucleotide primers. In a single PCR reaction a degenerated frameworks (FR1) primer ABL013 (5′-GAGGTBCARCTGCAGGASTCYGG-3′) was combined with a short (5′-AACAGTTAAGCTTCCGCTTGCGGCCGCGGAGCTGGGGTCTTCGCTGTGGTGCG-3′) or long (5′-AACAGTTAAGCTTCCGCTTGCGGCCGCTGGTTGTGGTTTTGGTGTCTTGGGTT-3′) hinge primer known to be specific for the amplification of heavy-chain variable region gene segments.

A PstI (bold) and Notl (bold underlined) restriction site was introduced within the FR1 and hinge primers respectively, to allow ,cloning. Subsequently, the DNA fragments were ligated into the PstI-NotI digested phagemid vector pAX004, which is identical to pHEN1 (Hoogenboom H R, et al. (1991). Multi-subunit proteins on the surface of filamentous phage: methodologies for displaying antibody (Fab) heavy and light chains. Nucleic Acids Res 19:4133-4137), but encodes a carboxyterminal (His)₆- and c-myc-tag for purification and detection, respectively. The ligation mixture was desalted on a Microcon filter (YM-50, Millipore) and electroporated into E. coli TG1 cells to obtain a library containing 1.8×10⁷ clones. The transformed cells were grown overnight at 37° C. on a single 20×20 cm plate with LB containing 100 μg/ml ampicillin and 2% glucose. The colonies were scraped from plates using 2×TY medium and stored at −80° C. in 20% glycerol.

As quality control the percentage of insert-containing clones was verified on 24 clones for each library by PCR using a combination of vector based primers. This analysis revealed that 95% of the clones contained a VHH encoding insert. The variability was examined by HinfI fingerprint analysis of the amplified VHH fragment of these 24 clones, thereby showing that all clones were indeed different.

### ii) Amplification of Repertoire with Oligo-dt Primer and One IgG-derived Primer

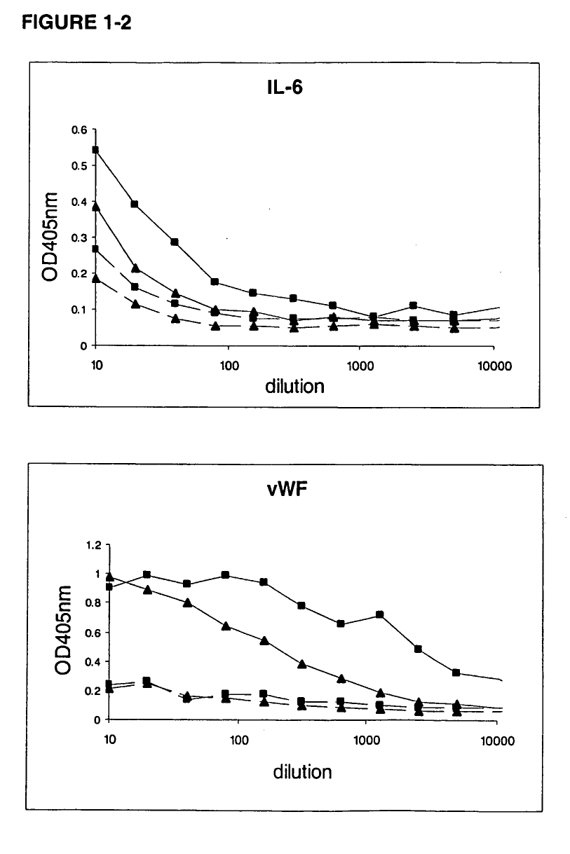

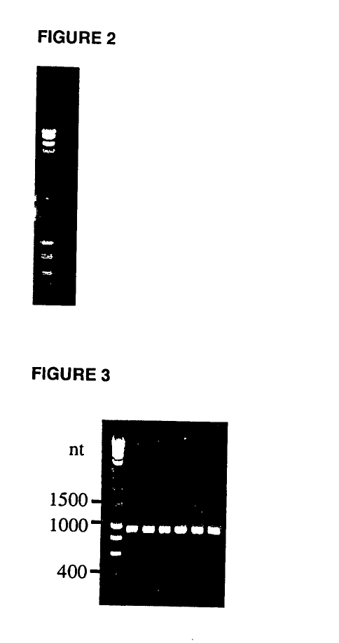

As template for PCR, oligo-dT primed cDNA was prepared on 100 μg of total RNA (de Haard et al., 1999). The VHH repertoire was amplified in three consecutive PCR amplifications as described in Example 1. PCR1 using oligo-dT and the primer that anneals to the immunoglobulin signal sequence results in the amplification of two fragments of 1650 bp and 1300 bp, the latter being the product derived from the CH1-deleted HcAb genes (see FIG. 2). This fragment was excised from gel and used for re-amplification with the oligo-dT primer, and a FR1 primer which introduced a NcoI-restriction site. The reamplified 1300 bp fragment was excised from gel and used in a third reamplification (PCR3) with the oligo-dT primer, and primer A4short which introduced a SfiI-restriction site. Approximately 10 μg of amplified VHH-harboring fragments were doubly digested with SfiI-BstEII. By agarose gelelectrophoresis, we estimated that more than 90% of the PCR3 product contained an internal BstEII restriction site (see FIG. 3).

In a second strategy a set of FR1 primers (Table 1), introducing a SfiI and NcoI restriction site, were used directly in combination with the oligo-dT primer thereby circumventing the re-amplification steps.

TABLE 1

Set of FR1 primers for camelid VHH amplification

| | |
|---|---|
| Name | Sequence (5′ - 3′) |

| | |
|---|---|
| ABL037 | CATGCCATGACTCGCGGCCCAGCCGGCCATGGCCGAGGTGCAGC |
| | TGGTGGAGTCTGG |
| ABL038 | CATGCCATGACTCGCGGCCCAGCCGGCCATGGCCGATGTGCAGC |
| | TGGTGGAGTCTGG |
| ABL039 | CATGCCATGACTCGCGGCCCAGCCGGCCATGGCCGCGGTGCAGC |
| | TGGTGGAGTCTGG |
| ABL040 | CATGCCATGACTCGCGGCCCAGCCGGCCATGGCCGCCGTGCAGC |
| | TGGTGGATTCTGG |
| ABL041 | CATGCCATGACTCGCGGCCCAGCCGGCCATGGCCCAGGTGCAGC |
| | TGGTGGAGTCTGG |
| ABL042 | CATGCCATGACTCGCGGCCCAGCCGGCCATGGCCCAGGTACAGC |
| | TGGTGGAGTCTGG |
| ABL043 | CATGCCATGACTCGCGGCCCAGCCGGCCATGGCCCAGGTAAAGC |
| | TGGAGGAGTCTGG |

Alternatively, a single degenerated FR1 primer ABL013 was used in combination with the oligo-dT primer to amplify the llama VHH repertoire. Single step PCR amplifications to recover the llama VHH repertoire were performed as described in PCR1 of example 1. The gel purified PCR products were digested with SfiI (or PstI when ABL013 was used) and BstEII. The BstEII-site frequently occurs within the FR4 of heavy-chain derived VHH encoding DNA-fragments as >90% of the purified PCR product was internally digested with BstEII.

300 ng of SfiI-BstEII digested fragments was ligated in the phagemid vector pAX004. The ligation reaction was incubated for 16 hours at room temperature using 10 units of T4 DNA ligase (Promega) in a total reaction volume of 300 μl. After adding two extra ligase units and subsequent incubation for 2 more hours at room temperature, the ligation mixture was purified with a double phenol and a chloroform extraction followed by an ethanol precipitation. The precipitated DNA was additionally washed with 70% ethanol, air-dried and dissolved in 50 μl HPLC-grade water. The purified ligation mix was divided in five equal aliquots and independently electroporated into 200 μl of electrocompetent E. coli TG1 cells with the micropulser (Biorad) at 1.8 kV using five 0.2 cm cuvettes. The transformed cells in each cuvette were recovered with 1 ml of 2×TY. Selection of pAX004-containing TG1 cells was performed on a single 20×20 cm plate with LB medium containing 100 μg/ml ampicillin and 2% glucose to yield a library with 1.4×10⁷ clones. The same type of quality control was performed as in section i), showing that 100% of the clones contained an insert of the appropriate size and confirmed the presence of a diverse repertoire.

### c. Titration of Antigen-specific Phage

From both libraries described in sections b.i. and b.ii., phages were prepared. To rescue the polyclonal phage repertoire, libraries were grown to logarithmic phase (OD600=0.5) at 37° C. in 2×TY containing 100 μg/ml ampicillin and 2% glucose and subsequently superinfected with M13K07 helper phages for 30 minutes at 37° C. Infected cells were pelleted for 5 minutes at 4000 rpm and resuspended in 2×TY containing 100 μg/ml ampicillin and 25 μg/ml kanamycin. Virions were propagated by overnight incubation at 37° C. and 250 rpm. Overnight cultures were centrifuged for 15 minutes at 4500 rpm and phages were precipitated in one fifth volume of a [20% polyethyleneglycol, 1.5 M NaCl]-solution by a 30-minute incubation on ice. Phages were pelleted by centrifugation for 15 minutes at 4000 rpm and 4° C. After resuspension of the phages in PBS, cell debris was pelleted by a 1-minute centrifugation at maximal speed in microcentrifuge tubes. The supernatant containing the phages was transferred to a new tube and again phages were precipitated as described above. The concentrated phages were dissolved in PBS and separated from remaining cell debris as mentioned above. The titer of phages was determined by infection of logarithmic TG1 cells followed by plating on selective medium. The titers of antigen-specific VHH fragments isolated from both libraries were compared by phage ELISA. Phages were applied to antigen coated (1 μg/ml) Maxisorp ELISA plates in duplo dilutions starting at 2×10¹⁰ phages/ml. Bound phages were detected by incubation with an anti-M13 horse radish peroxidase conjugate and subsequent development.

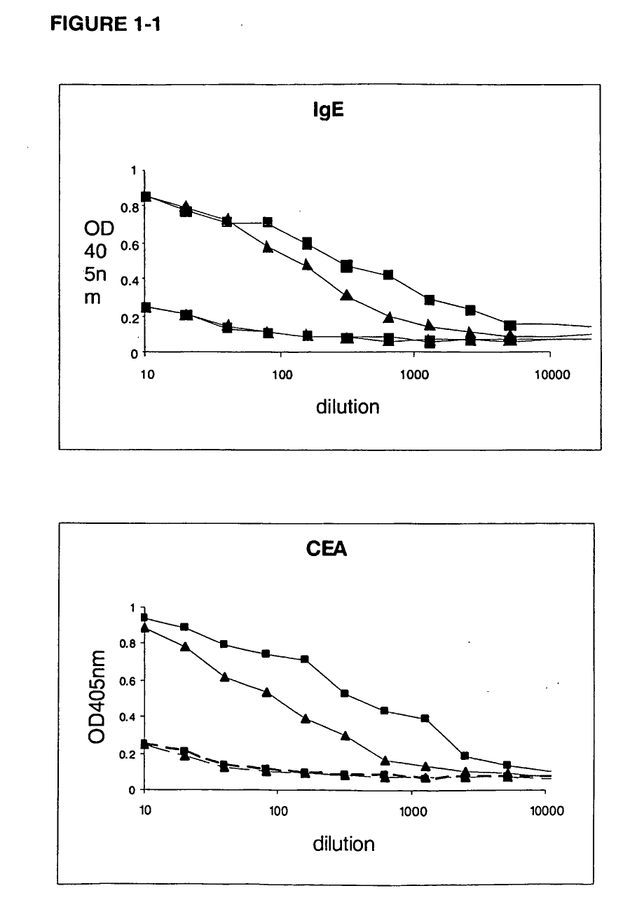

For all antigens tested, antigen specific phage titers were significantly higher when phages were rescued from the library expressing the repertoire amplified with a single IgG specific primer (FIG. 1).

### d. Selection and Screening of the Immune Library with Target Antigens

From the library described in section b.ii. (VHH repertoire amplified with a single species-specific primer and the oligo-dT primer), phages were rescued as described in section c. Antigen-specific binders were selected using the principle of phage display and a single round of biopanning on solid phase coated TNFα, vWF, CEA or IL-6 at concentrations of 5 μg/ml (Marks J D, et al (1991) By-passing immunization. Human antibodies from V-gene libraries displayed on phage. J. Mol. Biol. 222, 581-597., 1991; Hawkins R E, et al (1992) Selection of phage antibodies by binding affinity. Mimicking affinity maturation. J. Mol. Biol. 226, 889-896). After a 2 hour incubation of rescued phages with the respective immobilized antigens, the non-specific phages were washed away, while specific phages were eluted for 20 minutes with a pH shock (0.1 M glycine pH2.5) and subsequently neutralized with 1M Tris buffer pH 7.5. Log phase growing E. coli cells were infected with the eluted phages and plated on selective medium. For each antigen, 48 clones were picked and further characterized.

Culture supernatant of these 48 individual clones was prepared by growing them until log phase in 2×TY containing 100 μg/ml ampicillin and 0.1% glucose. Subsequently, the expression of VHH-geneIII fusion proteins was induced with 1 mM IPTG followed by overnight incubation at 37° C. and 250 rpm. The specificity of VHH-expressing clones was verified in ELISA in antigen coated (1 μg/ml) versus non coated microwells (background) using crude culture supernatant. Signals that were twice the background after 20 minutes development were considered as positive and retained for further characterization.

### e. Evaluation of the Diversity of Repertoire Cloning Methods

From the library described in section b.i. (repertoire amplified with two immunoglobulin specific primers), phages were rescued as described in section c. Antigen-specific binders against IgE and CEA were selected on solid phase coated immunotubes (5 μg/ml) by a single round of panning under identical conditions as described in section d. After screening the supernatant of 48 individual clones in ELISA as described above and subsequent sequencing of the representative clones corresponding to all identified different HinfI profiles, 12 out of the 14 anti IgE-binders and 6 of 8 anti-CEA binders that were isolated from the libraries made with only one IgG specific primer could not be identified from the library made by using two IgG specific primers.

### 3. Human Immunoglobulin Repertoire Amplification

### a. Amplification of a Human Immunoglobulin Repertoire

Blood of two human donors was obtained from the bloodbank of the Belgian Red Cross. PBLs were isolated and total RNA was prepared. Hundred μg of total RNA was used for oligo-dT primed cDNA synthesis (de Haard et al., 1999) subsequently applied as template for immunoglobulin heavy and light chain amplification.

The human VH repertoire was amplified by using oligo-dT in combination with 5 different (sets of) oligonucleotides (Table 2) annealing to the FR1 of the distinct families of human VH genes.

TABLE 2

FR1 primers used for human
immunoglobulin VH amplification

| | |
|---|---|
| Name | Primer sequence 5′-3′ |

| | |
|---|---|
| Set 1 | CAGRTGCAGCTGGTGCARTCTGG |
| | SAGGTCCAGCTGGTRCAGTCTGG |
| Set 2 | SAGGTGCAGCTGGTGGAGTCTGG |
| | GARGTGCAGCTGGTGCAGTCTGG |
| Set 3 | CAGSTGCAGCTGCAGGAGTCSGG |
| | CAGGTACAGCTGCAGCAGTCAGG |
| Primer 4 | CAGRTCACCTTGAAGGAGTCTGG |
| Primer 5 | CAGGTGCAGCTGCAGCAGTGGGG |

When applying identical conditions as described for PCR1 (see Example 1), a fragment of approximately 1.6 kb was amplified for each combination of primers, corresponding to the expected size of IgG molecules. In the same amplification reaction an additional fragment of approximately 2.1 kb, corresponding to the calculated size of IgM amplification product, was also synthesized. To verify whether the 1.6 kb and 2.1 kb fragments correspond to IgG and IgM respectively, 1 ng of each gel purified fragment was reamplified by a nested PCR. The conditions of the amplification reaction were identical to PCR2 (Example 1) using the appropriate (set of) FR1 primers and an IgG-(5′-GTCCACCTTGGTGTTGCTGGGCTT-3′) or IgM-specific primer (5′-TGGAAGAGGCACGTTCTTTTCTTT-3′) that anneals to the CH1 domain. Indeed, when using the 1.6 kb gel purified fragment as template, a single fragment with expected size of 0.65 kb could only be amplified using the appropriate FR1 in combination with an IgG-specific but not an IgM-specific CH1 primer. On the contrary, using the 2.1 kb gel purified fragment as template, a single fragment with expected size of 0.67 kb was amplified using the appropriate FR1 in combination with an IgM-specific CH1 primer. As expected, the combination of the FR1 and an IgG-specific CH1 primer did not yield any PCR product. The gel purified 1.6 kb (or 2.1 kb) fragment was incubated with BstEII, resulting in the presence of two extra fragments of 0.38 (0.38) and 1.22 (1.72) kb after agarose gel electrophoresis. The presence of a unique BstEII restriction site in 5 of the 6 human J-genes indicates that the 1.6 and 2.1 kb fragments correspond to IgG and IgM, respectively. Based on the amount of undigested fragment, we estimate that >90% of the IgG or IgM amplification products carry an internal BstEII restriction site, making it a suitable candidate for VH repertoire cloning. The VH repertoire can be reamplified with oligo-dT combined with a set of FR1 primers introducing a unique restriction site such as SfiI that can be used for VH repertoire cloning.

The possibility to amplify the human VL repertoire was demonstrated in a PCR using oligo-dT primed cDNA applying the conditions as described for PCR1 (see Example 1). To maximize the recovery of repertoire diversity, respectively 6 and 4 sets of primers (Table 3) in combination with oligo-dT were used to amplify the Vλ and Vκ repertoire. All primer combinations resulted in the amplifications of a fragment of expected size of approximately 0.82 kb.

TABLE 3

FR1 primers used for human
immunoglobulin VL amplification

| | |
|---|---|
| Name | Primer sequence 5′-3′ |

| | |
|---|---|
| Vλ amplification | |
| Set 1 | CAGTCTGTGYTGACKCAGCCRCC |
| | CWGCCTGTGCTGACTCAGCCMCC |
| | CAGTCTGCCCTGACTCAGCCT |
| Primer 2 | CAGCYTGTGCTGACTCAATCRYC |
| Set 3 | CAGGCTGTGCTGACTCAGCCGKC |
| | CAGGCAGGGCTGACTCAGCCACC |
| Primer 4 | TCCTATGAGCTGACWCAGCCACC |
| Primer 5 | AATTTTATGCTGACTCAGCCCCA |
| Set 6 | CAGRCTGTGGTGACYCAGGAGCC |
| | TCTTCTGAGCTGACTCAGGACCC |
| Vκ amplification | |
| Set 1 | GAAATTGTGWTGACRCAGTCTCC |
| | GAAATTGTGCTGACTCAGTCTCC |
| Set II | GATGTTGTGATGACTCAGTCTCC |
| | GAYATYGTGATGACCCAGWCTCC |
| Primer III | GACATCCAGWTGACCCAGTCTCC |
| Primer IV | GAAACGACACTCACGCAGTCTCC |

TABLE 4

List of oligonucleotides

| SEQ | |
|---|---|
| ID NO: | SEQUENCE |
| 1 | GGCTGAGCTCGGTGGTCCTGGCT |
| 2 | CCAGCCGGCCATGGCTGATGTGCAGCTGGTGGA |
| | GTCTGG |
| 3 | CCAGCCGGCCATGGCTCAGGTGCAGCTGGTGGA |
| | GTCTGG |
| 4 | CATGCCATGACTCGCGGCCCAGCCGGCCATGGC |
| 5 | GAGGTBCARCTGCAGGASTCYGG |
| 6 | AACAGTTAAGCTTCCGCTTGCGGCCGCGGAGCT |
| | GGGGTCTTCGCTGTGGTGCG |
| 7 | AACAGTTAAGCTTCCGCTTGCGGCCGCTGGTTG |
| | TGGTTTTGGTGTCTTGGGTT |
| 8 | CATGCCATGACTCGCGGCCCAGCCGGCCATGGC |
| | CGAGGTGCAGCTGGTGGAGTCTGG |
| 9 | CATGCCATGACTCGCGGCCCAGCCGGCCATGGC |
| | CGATGTGCAGCTGGTGGAGTCTGG |
| 10 | CATGCCATGACTCGCGGCCCAGCCGGCCATGGC |
| | CGCGGTGCAGCTGGTGGAGTCTGG |
| 11 | CATGCCATGACTCGCGGCCCAGCCGGCCATGGC |
| | CGCCGTGCAGCTGGTGGATTCTGG |
| 12 | CATGCCATGACTCGCGGCCCAGCCGGCCATGGC |
| | CCAGGTGCAGCTGGTGGAGTCTGG |
| 13 | CATGCCATGACTCGCGGCCCAGCCGGCCATGGC |
| | CCAGGTACAGCTGGTGGAGTCTGG |
| 14 | CATGCCATGACTCGCGGCCCAGCCGGCCATGGC |
| | CCAGGTAAAGCTGGAGGAGTCTGG |
| 15 | CAGRTGCAGCTGGTGCARTCTGG |
| 16 | SAGGTCCAGCTGGTRCAGTCTGG |
| 17 | SAGGTGCAGCTGGTGGAGTCTGG |
| 18 | GARGTGCAGCTGGTGCAGTCTGG |
| 19 | CAGSTGCAGCTGCAGGAGTCSGG |
| 20 | CAGGTACAGCTGCAGCAGTCAGG |
| 21 | CAGRTCACCTTGAAGGAGTCTGG |
| 22 | CAGGTGCAGCTGCAGCAGTGGGG |
| 23 | GTCCACCTTGGTGTTGCTGGGCTT |
| 24 | TGGAAGAGGCACGTTCTTTTCTTT |
| 25 | CAGTCTGTGYTGACKCAGCCRCC |
| 26 | CWGCCTGTGCTGACTCAGCCMCC |
| 27 | CAGTCTGCCCTGACTCACCCT |
| 28 | CAGCYTGTGCTGACTCAATCRYC |
| 29 | CAGGCTGTGCTGACTCAGCCGKC |
| 30 | CAGGCAGGGCTGACTCAGCCACC |
| 31 | TCCTATGAGCTGACWCAGCCACC |
| 32 | AATTTTATGCTGACTCAGCCCCA |
| 33 | CAGRCTGTGGTGACYCAGGAGCC |
| 34 | TCTTCTGAGCTGACTCAGGACCC |
| 35 | GAAATTGTGWTGACRCAGTCTCC |
| 36 | GAAATTGTGCTGACTCAGTCTCC |
| 37 | GATGTTGTGATGACTCAGTCTCC |
| 38 | GAYATYGTGATGACCCAGWCTCC |
| 39 | GACATCCAGWTGACCCAGTCTCC |
| 40 | GAAACGACACTCACGCAGTCTCC |

1. A method for cloning polynucleotide sequences encoding immunoglobulin variable domains (IGVD):
(a) providing a sample comprising mRNA,
(b) carrying out a first strand cDNA synthesis using a universal primer,
(c) carrying out a second strand DNA synthesis using a first primer capable of hybridizing to a site at or adjacent to the 3′ end of each of the IGVD sequences on the anti-sense strand so producing double stranded DNA,
(d) cleaving the double stranded DNA with a restriction enzyme specific for a restriction site positioned such that cleavage with the restriction enzyme directed thereto produces double stranded DNA encoding a functional IGVD fragment, and
(e) cloning the resulting variable domain fragment sequences into a vector.

2. The method according to claim 1, wherein the double stranded DNA produced in step (c) is subsequently amplified using said first primer and said universal primer.

3. The method according to claim 1, wherein step (c) is an amplification step comprising use of said first primer and said universal primer, and the product of step (b) as the template.

4. The method according to claim 1, wherein the universal primer comprises the sequence of oligo-dT.

5. The method according to claim 1, wherein the universal primer comprises the sequence of a set of random primers.

6. The method according to claim 1, wherein said first primer encodes for at least one enzyme restriction site.

7. The method according to claim 1, wherein said sample comprises mRNA derived from lymphocytes.

8. The method according to any of claim 1, wherein the restriction site of step (d) is BstEII.

9. The method according to any of claim 1, wherein said mRNA is derived from humans.

10. The method according to claim 1, wherein said mRNA is derived from camelids.

11. The method according to claim 1, wherein said vector is an expression vector able to express at least part of IGVD polynucleotide sequences.

12. The method according to claim 1, wherein said IGVD polynucleotide sequences are heavy chain variable domain polynucleotide sequences.

13. The method according to claim 1, wherein said IGVD polynucleotide sequences are light chain variable domain polynucleotide sequences.

14. The method according to claim 1, wherein said IGVD polynucleotide sequences are heavy chain variable domain and light chain variable domain polynucleotide sequences.

15. An expression library obtainable by a method according to claim 1 comprising a repertoire of IGVD polynucleotide sequences.

16. An expression library obtained by a method according to claim 1 comprising a repertoire of IGVD polynucleotide sequences.

17. An IGVD polynucleotide obtainable according to the methods claim 1.

18. An IGVD polynucleotide obtained according to the method of claim 1.

19. A diagnostic assay based on the use of an expression library according to claim 15.

20. A diagnostic report obtained from the diagnostic assay according to claim 19.

21. A method of using a polypeptide obtained after expression of one of the cloned sequences of claim 1 for the manufacture of a medicament.

22. A diagnostic assay based upon the use of an IGVD polynucleotide according to claim 17.

23. A diagnostic report obtained from the diagnostic assay according to claim 22.
